(19) United States
(12) Patent Application Publication
(10) Pub. No.: US 2025/0186212 A1
(43) Pub. Date: Jun. 12, 2025

(54) LOW-CRYSTALLINE CALCIUM PHOSPHATE-BASED BONE GRAFT MATERIAL AND METHOD FOR PREPARING THE SAME

(71) Applicant: OSSTEMIMPLANT CO., LTD., Seoul (KR)

(72) Inventor: Hyungjoon PARK, Incheon (KR)

(21) Appl. No.: 18/730,770

(22) PCT Filed: Dec. 19, 2022
(86) PCT No.: PCT/KR2022/020746

§ 371 Date: Feb. 17, 2025

(30) Foreign Application Priority Data
Jan. 21, 2022 (KR) 10-2022-0009348

(51) Int. Cl. A61F 2/30; A61F 2/28

(52) U.S. Cl. CPC A61F 2/30942; A61F 2/28; A61F 2/30771; A61F 2002/2835; A61F 2310/00293

(57) ABSTRACT

Disclosed are a low-crystalline calcium phosphate-based bone graft material and a method of preparing same, the bone graft material comprising at least one of dents and pores having an average diameter of 100 to 500 μm, having a BET specific surface area of 100 m²/g or more, comprising both crystalline apatite and amorphous calcium phosphate, and having a degree of crystallization of 25% or less.

### TECHNICAL FIELD

The present specification relates to a low-crystalline calcium phosphate-based bone graft material and a method of preparing the same.

### BACKGROUND ART

Bone graft material is a material used to regenerate bone that has been absorbed or destroyed due to factors such as diseases, trauma, aging, and the like. For example, during dental treatment such as implant placement surgery, periodontal surgery, or the like, osteogenesis surgery is often performed to artificially create alveolar bone using the bone graft material.

Bone graft materials play a role in stimulating bone growth, and can be classified as osteoconductive, osteoinductive, and osteogenic depending on their mechanism of action. Osteoconduction is a mechanism by which osteoblasts migrate from surrounding bone tissue to form bone. In this case, bone tissue or differentiated mesenchymal cells at the transplant site are essential for osteoconduction. Osteoinduction refers to a mechanism by which bone tissue is formed by influencing the differentiation of undifferentiated mesenchymal cells into osteoprogenitor cells. Osteogenesis refers to a mechanism of generating bone tissue by cells inside the graft material.

Bone graft materials can be classified into autogenous bone, allogenic bone, heterogeneous bone, and alloplastic bone according to their origins. Autogenous bone graft materials use bone tissue collected from a recipient of transplantation, have all of osteogenic, osteoconductive, and osteoinductive abilities, and are the only existing bone graft materials to have osteogenic ability. However, an additional surgical procedure is required to collect bone tissue, which requires cost and time. Above all, there is a limitation in that an amount of bone that can be used is limited. Allogenic bone graft materials are obtained by collecting bone tissue from a cadaver, and are generally obtained by donating bone from a deceased person. Because this graft material is obtained from a genetically identical species, it has an equivalent level of healing ability and has excellent bone replacement effects and osteogenic ability due to absorbability. Heterogeneous bone graft materials are collected from individuals of different species, and mainly use hydroxyapatite obtained from bovine or porcine species. A variety of ingredients, including calcium phosphate-based ceramics, are used as alloplastic bone graft materials, but they are generally known to have inferior osteogenic ability and bone quality compared to the autogenous bone or allogenic bone graft materials.

Calcium phosphate-based biomaterials mainly used as alloplastic bone graft materials include hydroxyapatite (HA) or tricalcium phosphate (TCP). However, hydroxyapatite has a disadvantage of remaining in the body for a long period of time due to its non-absorbability, and tricalcium phosphate has a problem of an imbalance between the formation rate of bone and the decomposition rate of the graft material.

Accordingly, there is a need to develop an alloplastic bone graft material that has an osteogenic ability similar to that of the allogenic bone while exhibiting absorbability.

### Technical Problem

The description in this specification is intended to solve the problems of the related art as described above, and therefore the object of the present specification is to provide an osteoconductive bone graft material having excellent osteogenic ability and a method of preparing the same.

### Technical Solution

According to one aspect, there is provided a low-crystalline calcium phosphate-based bone graft material, which includes at least one of dents and pores having an average diameter of 100 to 500 μm, has a BET specific surface area of 100 m²/g or more, includes both crystalline apatite and amorphous calcium phosphate, and has a degree of crystallization of 25% or less.

According to one embodiment, the integrated intensity I_{c} of the bone graft material at a wavenumber of 625 to 635 cm⁻¹ corresponding to an apatite structure, which is a crystalline phase, may be less than or equal to the integrated intensity Iₐ at a wavenumber of 530 to 540 cm⁻¹ corresponding to a calcium phosphate structure, which is an amorphous phase, as measured by an FT-IR method.

According to one embodiment, the 3-week volume (V₃) of the bone graft material and the 12-week volume (V₁₂) of the bone graft material may satisfy the following Expression 1 in a rabbit calvarial defect model:

$$0.2 \leq ( {V}_{3} - {V}_{12} ) / {V}_{3} \leq 0.6 . \qquad [ Expression 1 ]$$

According to one embodiment, the bone graft material may have osteoconductive ability.

According to another aspect, there is provided a method of preparing a low-crystalline calcium phosphate-based bone graft material, which includes: (a) adding a calcium precursor and a phosphate precursor in a batchwise manner to synthesize calcium phosphate; (b) freeze-drying the synthesized calcium phosphate; (c) mixing the calcium phosphate with a shape control agent and pressure-molding the mixture to prepare a molded body; and (d) removing the shape control agent from the molded body in a solvent at a temperature above the boiling point and then drying the molded body.

According to one embodiment, the calcium precursor may include one or more selected from the group consisting of calcium nitrate, calcium chloride, calcium fluoride, calcium iodide, calcium acetate, ammonium carbonate, and ammonium bicarbonate.

According to one embodiment, the phosphate precursor may include one or more selected from the group consisting of phosphorus oxide, monobasic ammonium phosphate, dibasic ammonium phosphate, tribasic ammonium phosphate, monobasic sodium phosphate, dibasic sodium phosphate, tribasic sodium phosphate, and potassium phosphate.

According to one embodiment, the molar ratio of the calcium precursor and the phosphate precursor in step (a) may range from 1:1.5 to 3.

According to one embodiment, step (a) may be a wet precipitation method in which each precursor is dissolved and mixed in distilled water.

According to one embodiment, the weight ratio of the calcium phosphate and the shape control agent in step (c) may range from 1:1 to 1.5.

According to one embodiment, the shape control agent may have an average particle size of 100 to 500 μm.

According to one embodiment, the method may further include, after step (d), (e) heat-treating the molded body at a temperature of 250° C. or lower.

### Advantageous Effects

A bone graft material according to one aspect of the present specification can exhibit absorbability and osteoconductivity, and can have osteogenic ability similar to that of allogenic bone.

It should be understood that the effects of one aspect of the present specification are not limited to the effects described above and include all effects that can be deduced from the configurations described in the detailed description of the present specification or the claims of this specification.

### DESCRIPTION OF DRAWINGS

FIG. 1 shows stereoscopic microscope and SEM images of a bone graft material according to one embodiment of the present specification.

FIG. 2 shows SEM images of the bone graft material according to one embodiment of the present specification.

FIG. 3 shows the results of XRD analysis of the bone graft material according to one embodiment of the present specification.

FIG. 4 shows the results of XRD analysis of the bone graft material according to one embodiment of the present specification.

FIG. 5 shows the results of analyzing the BET specific surface area of the bone graft material according to one embodiment of the present specification.

FIG. 6 shows the micro-CT analysis results of the bone graft material according to one embodiment of the present specification.

FIG. 7 shows the results of analyzing the absorption rate of the bone graft material in a rabbit calvarial defect model according to one embodiment of the present specification.

FIG. 8 shows the FT-IR analysis results of the bone graft material according to one embodiment of the present specification.

FIG. 9 shows the results of FT-IR deconvolution analysis of the bone graft material according to one embodiment of the present specification.

FIG. 10 shows the results of FT-IR deconvolution analysis of the bone graft material according to one embodiment of the present specification.

FIG. 11 shows a micro-CT image and the results of osteogenic ability analysis of the bone graft material in an adult dog alveolar bone defect model according to one embodiment of the present specification.

### MODE FOR INVENTION

Hereinafter, one aspect of the present specification will be described with reference to the accompanying drawings. However, it should be understood that the descriptions of the present specification can be implemented in various other forms and are not intended to be limited to the exemplary embodiments of the present specification. Also, in the drawings, descriptions of parts unrelated to the detailed description will be omitted to clearly describe one aspect of the present specification. Throughout the specification, like numbers refer to like elements.

Throughout the specification, when a certain element is referred to as being “connected to another element, it can be directly connected to the other element or “indirectly connected” to the other element with one or more intervening elements interposed therebetween. In addition, when an element is referred to as “including” another element, it should be understood that the element may further include still another element rather than excluding the still another element unless particularly indicated otherwise.

When a range of numerical values is presented in the present specification, the value has the precision of significant digits provided in accordance with the standard rules in chemistry for significant digits unless a specific range is stated otherwise. For example, the numerical value 10 includes the range of 5.0 to 14.9 and the numerical value 10.0 includes the range of 9.50 to 10.49.

Hereinafter, embodiments of the present specification will be described in detail with reference to the accompanying drawings.

### Low-Crystalline Calcium Phosphate-Based Bone Graft Material

A low-crystalline calcium phosphate-based bone graft material according to one aspect of the present specification may include at least one of dents and pores having an average diameter of 100 to 500 μm, have a BET specific surface area of 100 m²/g or more, include both crystalline apatite and amorphous calcium phosphate, and have a degree of crystallization of 25% or less.

In the present specification, “low crystallinity” may mean that the proportion of an apatite structure, which is a crystalline phase, among the constituent phases in the bone graft material, is lower than usual. The “degree of crystallization” may be calculated through X-ray diffraction (XRD) analysis according to the ISO 13779-3 standard. For example, a low-crystalline bone graft material may refer to a bone graft material having a degree of crystallization of less than half or 25% or less, but the present specification is not limited thereto. In this case, the low-crystalline bone graft material may refer to a low-crystalline component recognized within the typical technical scope known in the art.

The bone graft material may include concave dents or pores at the centers of particles. These dents or pores may promote the formation of new blood vessels and the growth of new bone by allowing blood cells, osteoblasts, osteoclasts, proteins, and growth factors to be supported in the bone graft material particles.

The dents or pores of the low-crystalline calcium phosphate-based bone graft material may have an average diameter of 100 to 500 μm. For example, the average diameter of the dents or pores may be 100 μm, 120 μm, 140 μm, 160 μm, 180 μm, 200 μm, 220 μm, 240 μm, 260 μm, 280 μm, 300 μm, 320 μm, 340 μm, 360 μm, 380 μm, 400 μm, 420 μm, 440 μm, 460 μm, 480 μm, 500 μm, or a value in a range between two of these values. When the average diameter of the dents or pores is outside the above range, the osteogenic ability of the bone graft material may deteriorate, or the specific surface area or crystallinity may be outside the desired range.

In the present specification, the “specific surface area” refers to a value calculated by a method based on the Brunauer, Emmett and Teller (BET) theory. In this case, this value is estimated by measuring an amount of gas (e.g., nitrogen) physically adsorbed on the surface of a sample and calculating the area required for the gas to be adsorbed on a smooth surface.

The low-crystalline calcium phosphate-based bone graft material may have a BET specific surface area of 100 m²/g or more. For example, the BET specific surface area may be 100 m²/g, 102 m²/g, 104 m²/g, 106 m²/g, 108 m²/g, 110 m²/g, 112 m²/g, 114 m²/g, 116 m²/g, 118 m²/g, 120 m²/g, 122 m²/g, 124 m²/g, 126 m²/g, 128 m²/g, 130 m²/g, 132 m²/g, 134 m²/g, 136 m²/g, 138 m²/g, 140 m²/g, 142 m²/g, 144 m²/g, 146 m²/g, 148 m²/g, 150 m²/g, or a value greater than or equal to any of these values or a value in a range between two of these values, but the present specification is not limited thereto. When the BET specific surface area is less than 100 m²/g, it may be difficult to bind and store blood cells, osteoblasts, osteoclasts, proteins or various growth factors. On the other hand, when the BET specific surface area satisfies the above range, blood clot formation may be easy, and the adhesion of osteogenesis-related cells may be excellent due to excellent blood wetting properties, thereby promoting bone fusion between the graft material and bone tissue.

Conventional alloplastic bone has the disadvantage of having a small surface area because particles grow and become densified during a high-temperature sintering process. On the other hand, natural bone has a large surface area due to the characteristics of its microstructure and is known to have excellent osseointegration properties. The bone graft material according to one aspect of the present specification may have excellent osseointegration properties because it has a high surface area even though it is synthetic bone.

The “crystalline apatite” may refer to a hexagonal calcium phosphate mineral. For example, the calcium phosphate mineral may be one or more selected from the group consisting of hydroxyapatite, fluorapatite, chlorapatite, and carbonate apatite.

As one example, the crystalline apatite may further include inorganic ions. The inorganic ions may be one or more selected from the group consisting of Mg²⁺, Sr²⁺, Ba²⁺, Mn²⁺, Zn²⁺, Na⁺, K⁺, Ag⁺, Ga³⁺, VO₄³⁻, SO₄³⁻, CO₃²⁻, SiO₄³⁻, F⁻, and Cl⁻, but the present specification is not limited thereto. The inorganic ions may be added during a process of synthesizing calcium phosphate from a calcium precursor and a phosphate precursor, but the present specification is not limited thereto.

The low-crystalline calcium phosphate-based bone graft material may include both an apatite structure, which is a crystalline phase and a calcium phosphate structure, which is an amorphous phase. The ratio of the apatite structure, which is a crystalline phase, and the calcium phosphate structure, which is an amorphous phase, may be analyzed using an FT-IR Voigt deconvolution method. The integrated intensity I_{c} of the bone graft material at a wavenumber of 625 to 635 cm⁻¹ may be less than or equal to the integrated intensity Iₐ at a wavenumber of 530 to 540 cm⁻¹, as measured by an FT-IR method. The integrated intensity I_{c} at a wavenumber of 625 to 635 cm⁻¹ may refer to the proportion of apatite-based OH⁻ corresponding to the apatite structure, which is a crystalline phase. The integrated intensity Iₐ at a wavenumber of 530 to 540 cm⁻¹ may refer to non-apatite-based HPO₄³⁻ corresponding to the calcium phosphate structure, which is an amorphous phase. Therefore, in the low-crystalline calcium phosphate-based bone graft material, the proportion of the calcium phosphate structure, which is an amorphous phase, may be greater than the proportion of the apatite structure, which is a crystalline phase. The ratio I_{c}/Iₐ of the bone graft material may be 1.0 or less, for example 1.0, 0.95, 0.90, 0.85, 0.80, 0.75, 0.70, 0.65, 0.60, 0.55, 0.50, or a value less than or equal to any of these values or a value in a range between two of these values, but the present specification is not limited thereto. When the ratio of the apatite structure, which is a crystalline phase, and the calcium phosphate structure, which is an amorphous phase, satisfies the above range, the bone graft material may be easily resorbed by osteoclasts during bone regeneration and replaced with new bone. This rate of absorption may be adjusted by controlling the degree of crystallization of the bone graft material.

The bone graft material may have a degree of crystallization of 25% or less. For example, the degree of crystallization of the bone graft material may be 25%, 24.5%, 24%, 23.5%, 23%, 22.5%, 22%, 21.5%, 21%, 20.5%, 20%, 19.5%, 19%, 18.5%, 18%, 17.5%, 17%, 16.5% 16%, 15.5%, 15%, 14.5%, 14%, 13.5%, 13%, 12.5%, or a value less than or equal to any of these values or a value in a range between two of these values, but the present specification is not limited thereto. When the degree of crystallization of the bone graft material is outside the above range, the absorption rate may be excessively fast or the bone graft material may not have absorbability.

### Method of Preparing Low-Crystalline Calcium Phosphate-Based Bone Graft Material

A method of preparing the low-crystalline calcium phosphate-based bone graft material may include: (a) adding a calcium precursor and a phosphate precursor in a batchwise manner to synthesize calcium phosphate; (b) freeze-drying the synthesized calcium phosphate; (c) mixing the calcium phosphate with a shape control agent and pressure-molding the mixture to prepare a molded body; and (d) removing the shape control agent from the molded body in a solvent at a temperature above the boiling point and then drying the molded body.

Step (a) may be a process of preparing raw materials for low-crystalline calcium phosphate-based bone graft material. Each precursor may form calcium phosphate by a precipitation method in which wet mixing is performed in a solution mixed with distilled water. The calcium precursor may include one or more selected from the group consisting of calcium nitrate, calcium chloride, calcium fluoride, calcium iodide, calcium acetate, ammonium carbonate, and ammonium bicarbonate. The phosphate precursor may include one or more selected from the group consisting of phosphorus oxide, monobasic ammonium phosphate, dibasic ammonium phosphate, tribasic ammonium phosphate, monobasic sodium phosphate, dibasic sodium phosphate, tribasic sodium phosphate, and potassium phosphate.

In the above-mentioned precipitation method, there is also a process using a method of adding precursors dropwise to maintain vacuum conditions. However, because the above preparation method does not require vacuum conditions as a prerequisite, the calcium phosphate may be synthesized simply by batchwise addition.

The molar ratio of the calcium precursor and the phosphate precursor in step (a) may be 1:1.5 to 3. For example, the molar ratio of the calcium precursor and the phosphate precursor may be 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, or a value in a range between two of these values. When the molar ratio is outside the above range, the reaction may proceed inefficiently or it may be difficult to manufacture a bone graft material with the desired composition.

Step (a) may be a wet precipitation method in which each precursor is dissolved and mixed in distilled water. In step (b), the solvent used in step (a) can be removed by freeze-drying the synthesized calcium phosphate. Before step (b), the calcium phosphate may be aged for 1 to 24 hours, filtered, frozen, and then used. The freeze drying may be a method of reducing atmospheric pressure at a low temperature below room temperature to remove moisture by sublimation. These freeze-drying conditions are not limited as long as the temperature and pressure allow ice to sublimate.

For example, the freeze-drying may be performed for 24 to 48 hours. Using freeze-drying, damage to or degeneration of the bone graft material may be minimized. When high-temperature drying is performed during drying of the calcium phosphate, the proportion of the apatite structure, which is a crystalline phase, increases, which makes it difficult to achieve the desired properties.

In step (c), a molded body may be prepared by mixing calcium phosphate and a shape control agent and pressure-molding the resulting mixture. The shape control agent may be, for example, sodium chloride (NaCl), but the present specification is not limited thereto. The shape control agent may function as a template to form the framework of the bone graft material during molding and hydrothermal treatment of calcium phosphate.

Shape control agents having an average particle size of 100 to 500 μm may be used as the shape control agent. For example, the average particle size may be 100 μm, 120 μm, 140 μm, 160 μm, 180 μm, 200 μm, 220 μm, 240 μm, 260 μm, 280 μm, 300 μm, 320 μm, 340 μm, 360 μm, 380 μm, 400 μm, 420 μm, 440 μm, 460 μm, 480 μm, 500 μm, or a value in a range between two of these values. The area from which the shape control agent has been removed may form dents or pores in the bone graft material. Therefore, when the average particle size of the shape control agent satisfies the above range, a low-crystalline calcium phosphate-based bone graft material including at least one of dents or pores having an average diameter of 100 to 500 μm may be prepared.

The weight ratio of the calcium phosphate and the shape control agent in step (c) may be 1:1 to 1.5. For example, the weight ratio of the calcium phosphate and the shape control agent may be 1:1, 1:1.05, 1:1.1, 1:1.15, 1:1.2, 1:1.25, 1:1.3, 1:1.35, 1:1.4, 1:1.45, 1:1.5, or a value in a range between two of these values, but the present specification is not limited thereto. When the weight ratio is outside the above range, it may be difficult to remove the shape control agent, or the pore or dents of the bone graft material may not be properly formed, which results in deteriorated bone regeneration and osteoconductive abilities.

Step (d) may be a process of removing the shape control agent acting as a template and imparting desired characteristics to the bone graft material. For example, a hydrothermal treatment process may be performed in water at a temperature of 100° C., which is greater than or equal to the boiling point, to remove the shape control agent present on the inside and outside of the molded body. Thereafter, the hydrothermally treated molded body may be dried under conditions of 250° C. or lower, 225° C. or lower, 200° C. or lower, 175° C. or lower, 150° C. or lower, 125° C. or lower, or 100° C. or lower to remove moisture.

After step (d), the method may further include: (e) heat-treating the molded body at a temperature of 250° C. or lower. The heat treatment may be, for example, performed at 250° C., 225° C., 200° C., 175° C., 150° C., 125° C., 100° C., 75° C., 50° C., or a temperature less than or equal to any of these temperatures or a temperature in a range between two of these temperatures. When the heat treatment temperature is greater than 250° C., the surface area may decrease and the degree of crystallinity may increase due to densification caused by grain growth. As a result, the absorbability and osteogenic ability of the bone graft material may deteriorate.

That is, by controlling the temperature in the heat treatment process, the surface area of the bone graft material may be increased to facilitate the binding and storage of proteins and growth factors, and the adhesion of cells related to osteogenic ability may increase by increasing blood wetting and blood clot formation. Ultimately, this may promote bone fusion between the graft material and bone tissue.

In addition, the absorption rate of the bone graft material may be controlled by adjusting the temperature in the heat treatment process. When the heat treatment is performed at a relatively high temperature, the degree of crystallization may increase, and thus the absorption rate may slow down.

Hereinafter, embodiments of the present specification will be described in more detail. However, it should be understood that the following experimental results describe only representative experimental results in the above embodiments, and the scope and content of the present specification should not be interpreted as being reduced or limited by the embodiments. Each effect of various implementations of the present specification that are not explicitly presented below will be described in detail in the corresponding sections.

### Example 1

A calcium ion solution was prepared by dissolving 0.3 M calcium nitrate tetrahydrate (Ca(NO₃)₂·4H₂O), which is a calcium precursor, in distilled water (H₂O). A phosphate ion solution was prepared by dissolving 0.6 M dibasic ammonium phosphate ((NH₄)₂HPO₄), which is a phosphate precursor, in distilled water whose pH was adjusted to 9.5 to 11.5 with ammonia water. A hydroxyapatite precipitate was prepared by mixing the calcium ion solution and the phosphate ion solution at a molar ratio of 1:2.

The hydroxyapatite precipitate was aged at room temperature for 12 hours, and then filtered and washed using a suction filter, a vacuum pump, and filter paper. Thereafter, the filtered hydroxyapatite precipitate was frozen. The frozen hydroxyapatite precipitate was freeze-dried for 36 to 48 hours to prepare hydroxyapatite powder.

Sodium chloride (NaCl), which is a shape control agent, was sieved to a size of 100 to 500 μm, and the hydroxyapatite powder and the sieved sodium chloride were dry mixed at a weight ratio of 1:1 to 1.5. The mixed powder was placed in a mold and pressed to prepare a molded body.

The prepared molded body was placed into distilled water, and heated for 2 to 4 hours to remove sodium chloride in the molded body, and the hydrothermally treated molded body was dried at 100° C. for 12 to 24 hours.

The dried molded body was pulverized to 0.25 to 2 mm to prepare particles. The particles were placed into a sieve, soaked in distilled water, and washed with running distilled water for 4 to 8 hours to remove impurities. The washed particles were dried at 100° C. for 12 to 24 hours to prepare a bone graft material.

### Example 2

A bone graft material was prepared in the same manner as in Example 1, except that the particles prepared by pulverizing the dried molded body were heat-treated at 200° C.

### Comparative Example 1

A bone graft material was prepared in the same manner as in Example 2, except that the heat treatment temperature was changed to 300° C.

### Comparative Example 2

A bone graft material was prepared in the same manner as in Example 2, except that the heat treatment temperature was changed to 400° C.

### Comparative Example 3

A bone graft material was prepared in the same manner as in Example 2, except that the heat treatment temperature was changed to 500° C.

### Experimental Example 1: Shape Analysis

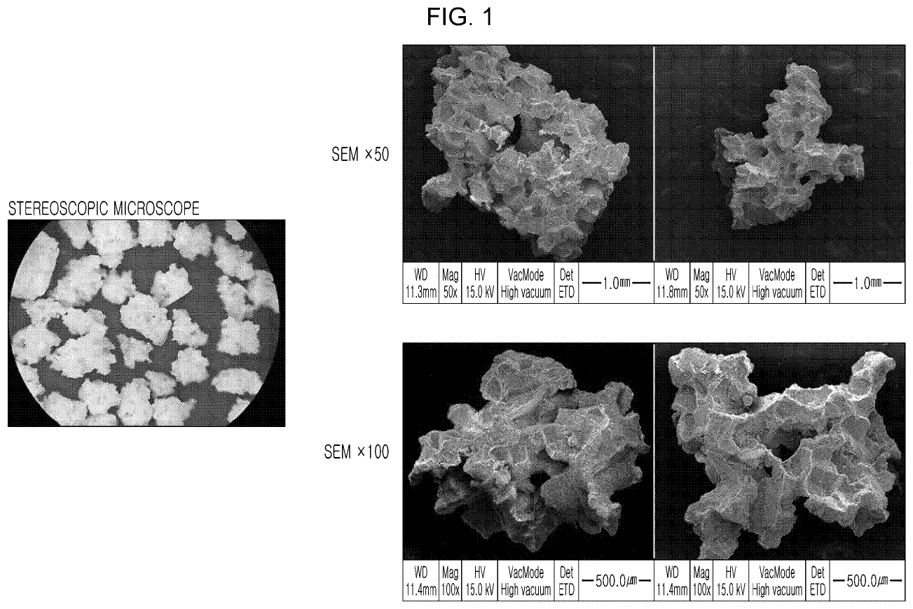

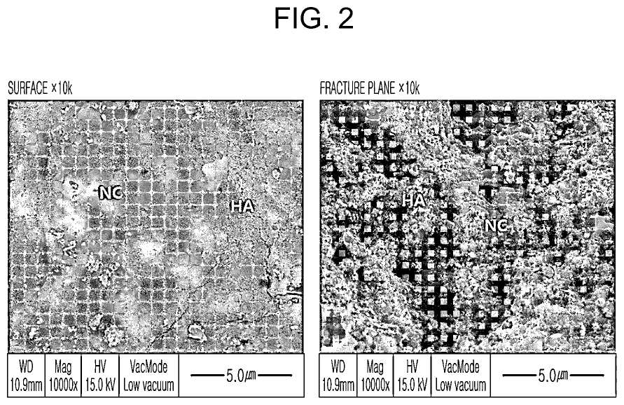

The bone graft material prepared according to Example 1 was photographed using a stereoscopic microscope and a scanning electron microscope (SEM). The results are shown in FIGS. 1 and 2. Referring to FIG. 1, it can be seen that dents (concavities) or pores (macropores) having a size of 100 to 500 μm were formed. Referring to FIG. 2, it can be seen that the nanoscale crystalline phase (HA) and the amorphous phase (i.e., non-crystalline phase (NC)) were mixed.

### Experimental Example 2: Analysis of Degree of Crystallization

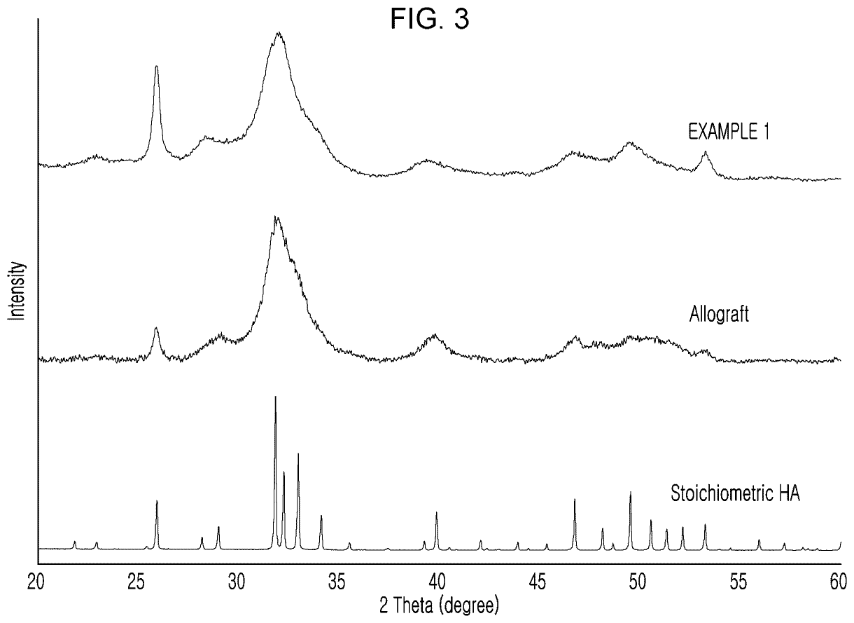

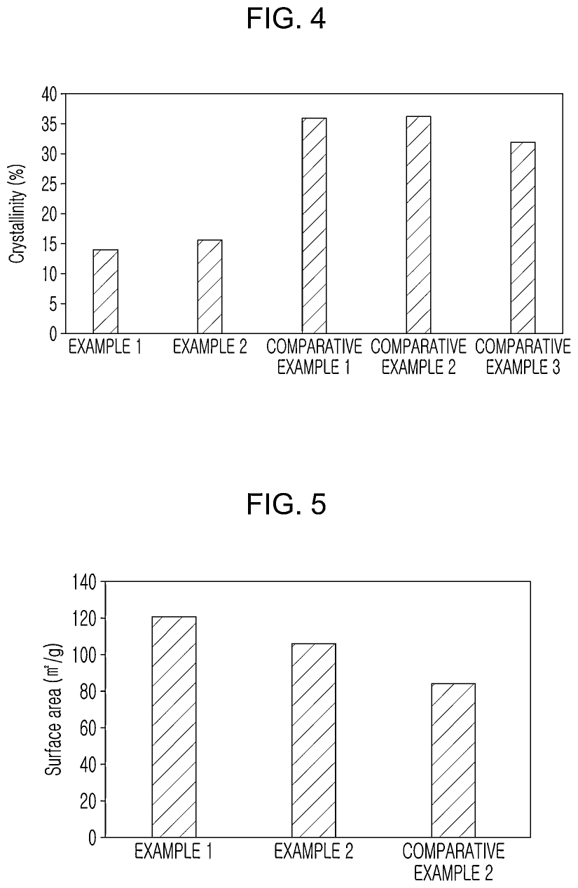

The bone graft material prepared according to Example 1 and allogenic bone (i.e., allograft) were subjected to X-ray diffraction (XRD) analysis according to the ISO 13779-3 standard. The results are shown in FIG. 3. The degree of crystallization calculated by substituting the values, which were obtained by subjecting Examples 1 and 2 and Comparative Examples 1, 2 and 3 to XRD analysis, into the following Equation 1 is shown in FIG. 4.

$$Degree of crystallization = \frac{Sum of peak intensities of 10 samples}{Sum of peak intensities of 10 HA standards} \times 100 \qquad 〈 Equation 1 〉$$

Referring to FIG. 3, it can be seen that Example 1 was composed of a low-crystalline HA component that is very similar to allogenic bone.

Referring to FIG. 4, it can be seen that the degree of crystallization varied depending on the heat treatment conditions. The degree of crystallization of Example 1, which was not heat-treated, was found to be 15.7%. As the heat treatment temperature increased, the degree of crystallization increased, but Comparative Example 3, in which the heat treatment temperature was 500° C., showed a slightly lower degree of crystallization of 36.3%. This may be assumed to be a decrease in degree of crystallization due to the phase change: HA→TCP(HPO₄²⁻+OH⁻→PO₄³⁻+H₂O).

### Experimental Example 3: Surface Area Analysis

The specific surface areas of the bone graft materials prepared according to Examples 1 and 2 and Comparative Example 2 were analyzed according to the BET method using nitrogen gas. The results are shown in FIG. 5. Example 1, which was not heat-treated, had the highest specific surface area of 121 m²/g, and Comparative Example 2, in which the heat treatment temperature was 400° C., had a specific surface area of 84 m²/g, indicating that the specific surface area decreased as the heat treatment temperature increased.

### Experimental Example 4: Absorbability Evaluation

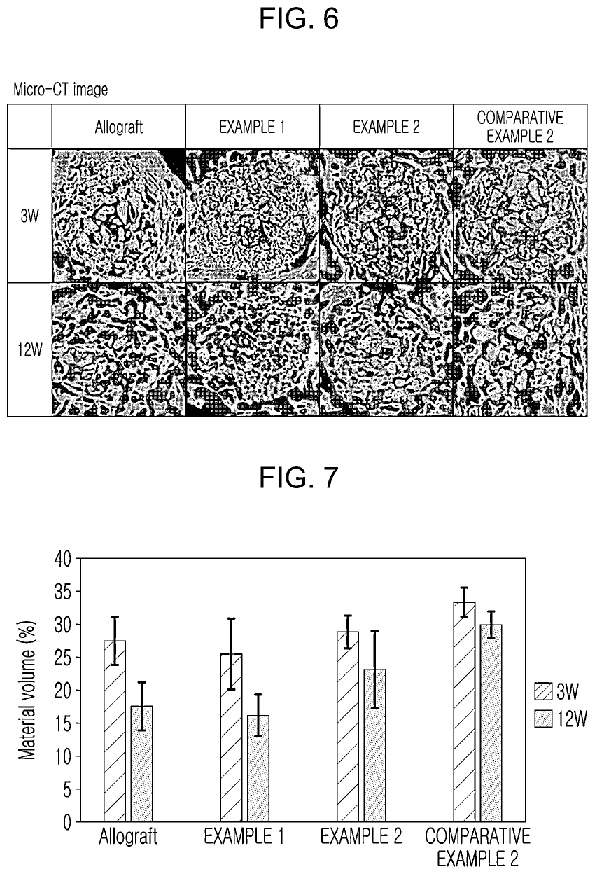

After the bone graft materials prepared according to Examples 1 and 2 and Comparative Example 2 were applied to a rabbit calvarial defect model, micro-CT images at weeks 3 and 12 were obtained and are shown in FIG. 6. A quantitative evaluation of the absorption rate was performed based on the reduction rate of the volume of the bone graft material before transplantation from the volume at weeks 3 and 12 analyzed through micro-CT. The results are shown in FIG. 7.

Referring to FIGS. 6 and 7, the bone graft material of Example 1, which was not heat-treated, showed a level of absorbability equivalent to that of allogenic bone (i.e., allograft). In particular, it was confirmed that the absorption rate measured by the volume reduction rate at weeks 3 and 12 was equivalent, i.e., 45% for the allogenic bone and 48% for Example 1. The absorption rate was 35% for Example 2, in which the heat treatment temperature was 200° C., and 13% for Comparative Example 2, in which the heat treatment temperature was 400° C., indicating that the absorption rate may be adjusted depending on the heat treatment temperature.

### Experimental Example 5: FT-IR Analysis

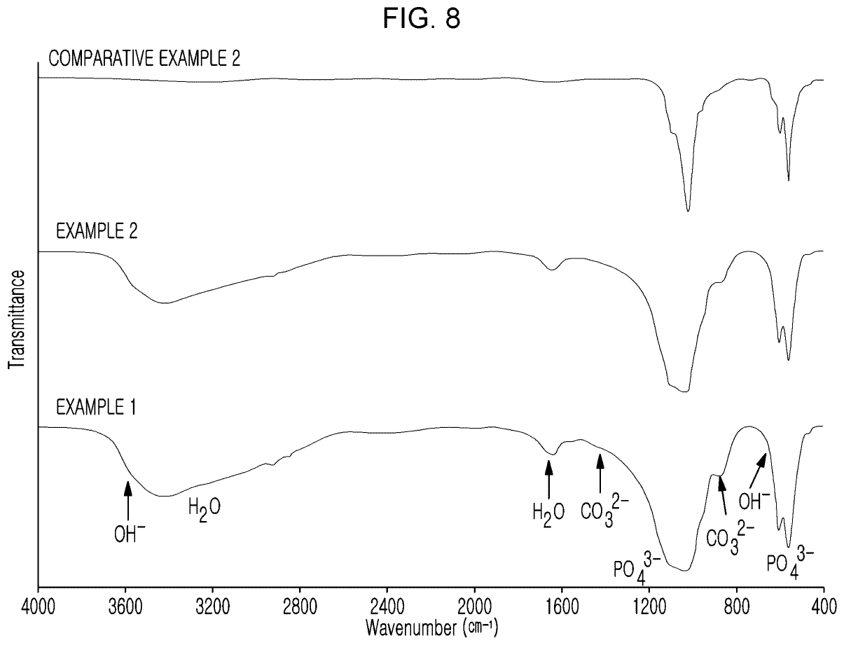

The bone graft materials prepared according to Examples 1 and 2 and Comparative Example 2 were analyzed by Fourier-transform infrared spectroscopy (FT-IR). The results are shown in FIG. 8.

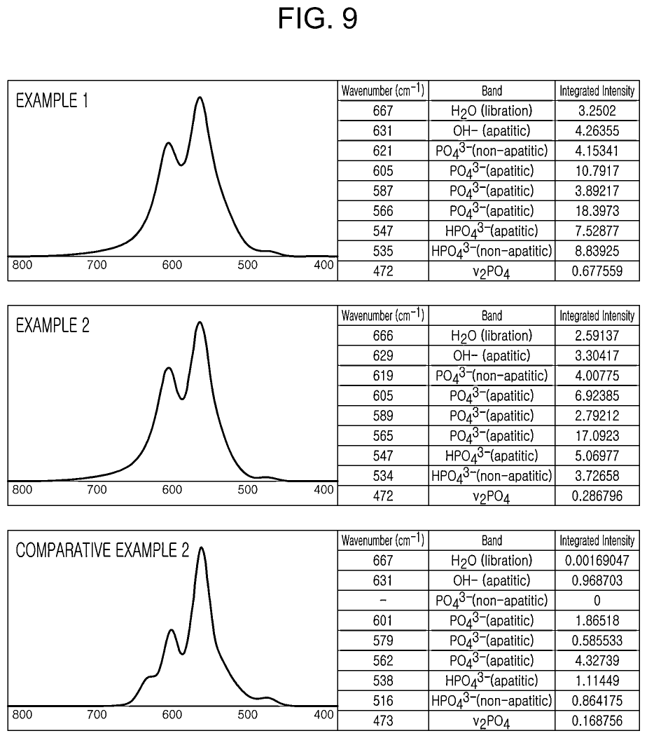

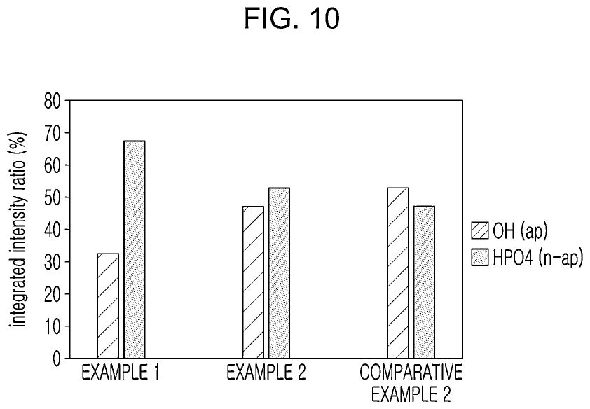

Referring to FIG. 8, it was confirmed that the bands tended to become sharper and the amorphous phase (i.e., non-crystalline phase (NC)) decreased as the heat treatment temperature increased. By deconvolving the spectrum in the wavenumber range of 400 to 800 cm⁻¹ corresponding to the ν₂νPO₄, ν_{L}OH region, the content of apatite-based OH-corresponding to the apatite structure, which is a crystalline phase, and the content of non-apatite-based HPO₄³⁻ corresponding to the calcium phosphate structure, which is an amorphous phase, were analyzed by a Voigt deconvolution method using Fityk 1.3.1 software. The results are shown in FIGS. 9 and 10.

Referring to FIGS. 9 and 10, as the heat treatment temperature increased, the integrated intensity ratio of the band at a wavenumber of 629 cm⁻¹ for apatite-based OH-increased. It can be seen that the integrated intensity ratio of the band at a wavenumber of 534 cm⁻¹ for non-apatite-based HPO₄³⁻ decreased as the heat treatment temperature increased. For example, it was found that the integrated intensity ratio of apatite-based OH⁻ was 33% in Example 1 and 53% in Comparative Example 2, and it was found that the integrated intensity ratio of non-apatite-based HPO₄³⁻ was 67% in Example 1 and 47% in Comparative Example 3. Therefore, it can be seen that the proportion of the apatite structure, which is a crystalline phase, increased compared to the calcium phosphate structure, which is an amorphous phase, at a heat treatment temperature of approximately 400° C.

### Experimental Example 6: Comparison with Allogenic Bone and Alloplastic Bone

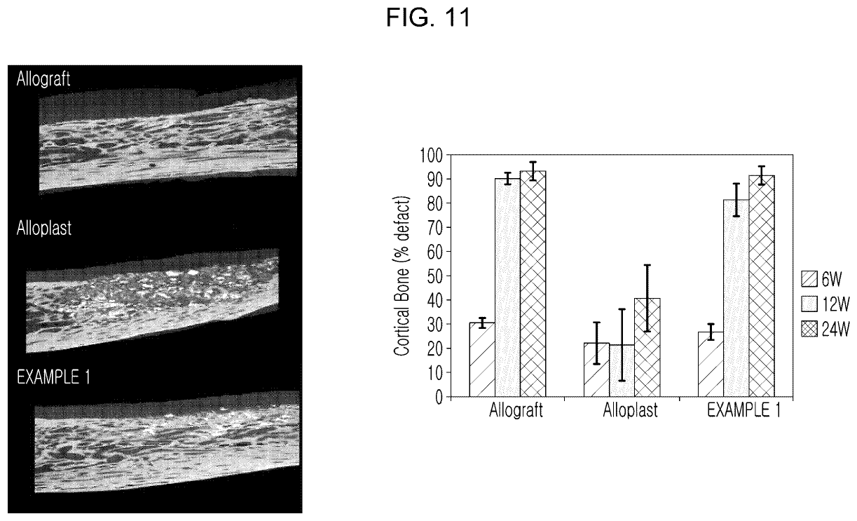

The bone graft material prepared according to Example 1, allogenic bone, and commonly used high-temperature sintered hydroxyapatite alloplastic bone were prepared, and their osteogenic abilities were compared through micro-CT imaging after 6, 12, and 24 weeks, respectively. The degree of cortical bone formation is shown in FIG. 11.

Referring to FIG. 11, it can be seen that Example 1 showed an osteogenic ability similar to allogenic bone at weeks 6, 12, and 24, and that the alloplastic bone had poor osteogenic ability compared to that of the allogenic bone.

The description of the present specification described above is for illustrative purposes, and it should be understood that those of ordinary skill in the art to which one aspect of the present specification belongs can easily modify it into other specific forms without changing the technical idea or essential features described in this specification. Therefore, it should be understood that the embodiments described above are illustrative in all respects and not restrictive. For example, each component described as a single type may be implemented in a distributed form, and likewise, components described as distributed may be implemented in a combined form.

The scope of the present specification is defined by the appended claims, and all changes or modifications derived from the meaning and scope of the claims and their equivalents should be construed as being included in the scope of the present specification.

1. A low-crystalline calcium phosphate-based bone graft material:
comprising at least one of dents and pores having an average diameter of 100 to 500 μm;
having a BET specific surface area of 100 m²/g or more;
including both crystalline apatite and amorphous calcium phosphate; and
having a degree of crystallization of 25% or less.

2. The low-crystalline calcium phosphate-based bone graft material of claim 1, wherein the integrated intensity I_{c} of the bone graft material at a wavenumber of 625 to 635 cm⁻¹ corresponding to an apatite structure, which is a crystalline phase, is less than or equal to the integrated intensity Iₐ at a wavenumber of 530 to 540 cm⁻¹ corresponding to a calcium phosphate structure, which is an amorphous phase, as measured by an FT-IR method.

3. The low-crystalline calcium phosphate-based bone graft material of claim 1, wherein the 3-week volume (V₃) of the bone graft material and the 12-week volume (V₁₂) of the bone graft material satisfy the following Expression 1 in a rabbit calvarial defect model:
0.2≤(V₃−V₁₂)/V₃≤0.6. [Expression 1]

4. The low-crystalline calcium phosphate-based bone graft material of claim 1, wherein the bone graft material has osteoconductive ability.

5. A method of preparing a low-crystalline calcium phosphate-based bone graft material, comprising:
(a) adding a calcium precursor and a phosphate precursor in a batchwise manner to synthesize calcium phosphate;
(b) freeze-drying the synthesized calcium phosphate;
(c) mixing the calcium phosphate with a shape control agent and pressure-molding the mixture to prepare a molded body; and
(d) removing the shape control agent from the molded body in a solvent at a temperature above the boiling point and then drying the molded body.

6. The method of claim 5, wherein the calcium precursor includes one or more selected from the group consisting of calcium nitrate, calcium chloride, calcium fluoride, calcium iodide, calcium acetate, ammonium carbonate, and ammonium bicarbonate.

7. The method of claim 5, wherein the phosphate precursor includes one or more selected from the group consisting of phosphorus oxide, monobasic ammonium phosphate, dibasic ammonium phosphate, tribasic ammonium phosphate, monobasic sodium phosphate, dibasic sodium phosphate, tribasic sodium phosphate, and potassium phosphate.

8. The method of claim 5, wherein the molar ratio of the calcium precursor and the phosphate precursor in step (a) ranges from 1:1.5 to 3.

9. The method of claim 5, wherein step (a) is a wet precipitation method in which each precursor is dissolved and mixed in distilled water.

10. The method of claim 5, wherein the weight ratio of the calcium phosphate and the shape control agent in step (c) ranges from 1:1 to 1.5.

11. The method of claim 5, wherein the shape control agent has an average particle size of 100 to 500 μm.

12. The method of claim 5, further comprising, after step (d):
(e) heat-treating the molded body at a temperature of 250° C. or lower.
